(19) United States
(12) Patent Application Publication
(10) Pub. No.: US 2011/0282348 A1
(43) Pub. Date: Nov. 17, 2011

(54) DEVICE FOR FIXATION OF BONE, AND DEVICE SET COMPRISING THE SAME

(75) Inventor: Min-Sik SHIN, Seoul (KR)

(73) Assignee: GS Medical Co., Ltd., Seoul (KR)

(21) Appl. No.: 12/948,299

(22) Filed: Nov. 17, 2010

(30) Foreign Application Priority Data
May 11, 2010 (KR) 10-2010-0043916

(51) Int. Cl. A61B 17/16; A61B 17/58

(52) U.S. Cl. 606/80; 606/93

(57) ABSTRACT

A device for fixation of bone, and more particularly, a device for forming a cavity in bone using an expandable member, is provided. The device for fixation of bone includes an external tube, an internal tube placed inside the external tube such that a fluid passage is formed between the internal tube and the external tube, the internal tube having open portions on both ends, and an expandable member surrounding one end of the internal tube, and connected by one and the other ends to the external tube and the internal tube, respectively, the expandable member in fluid communication with the fluid passage.

### CROSS-REFERENCE TO RELATED APPLICATIONS

This application claims priority from Korean Patent Application No. 10-2010-0043916, filed on May 11, 2010, in the Korean Intellectual Property Office, the disclosure of which is incorporated herein by reference in its entirety.

### BACKGROUND OF THE INVENTION

1. Field of the Invention

The present invention relates to a device for fixation of bone and a device set comprising the same, and more particularly, to a device for forming a cavity within bone using an expandable member and a device set comprising the same.

2. Description of the Related Art

Patients with osteoporosis, and particularly the aged patients are at increased risk of falls in which case even a slight shock can cause them tremendous pains in mainly spine or back. Such extreme pain is generally due to the ‘compression fracture’ in which the weak spinal bone is collapsed. The balloon kyphoplasty is widely used for treatment of the patients with the compression fracture.

The balloon kyphoplasty uses a narrow tube (i.e., catheter tube) with an expandable member such as a balloon formed on a leading end. That is, a surgeon injects the catheter tube into a spinal bone, inflates the balloon to create a void therein, and then injects orthopaedic cement into the cavity.

The balloon kyphoplasty is considered to be the effective treatment for not only the osteoporotic patients, but also patients with compression fractures from accident or trauma. The balloon kyphoplasty particularly reduces the risk of surgery due to use of general anesthesia, since the balloon kyphoplasty can be carried out with local anesthesia. Additionally, since the procedure does not involve bleeding, blood transfusion is not necessary, and the treatment is minimally invasive since it does not damage the neighboring areas such as normal muscle, bones, or nerves, or leave wounds or scars.

Meanwhile, conventionally, the kyphoplasty generally requires the use of a stylet which causes the structure to be complex and production cost to increase. Furthermore, it is necessary to separately remove the stylet during the balloon kyphoplasty, and the procedure is quite cumbersome.

### SUMMARY OF THE INVENTION

Exemplary embodiments of the present inventive concept overcome the above disadvantages and other disadvantages not described above. Also, the present inventive concept is not required to overcome the disadvantages described above, and an exemplary embodiment of the present inventive concept may not overcome any of the problems described above.

According to one embodiment, a device with a simple structure for fixation of bone, and more particularly, a device with a simple structure for fixation of bone, which forms a cavity within bone using an expandable member during balloon kyphoplasty, is provided.

In another embodiment, a device for fixation of bone which facilitates a procedure for bone or vertebral bone treatment, and a device set comprising the same, are provided.

According to an embodiment, a device for fixation of bone may include an external tube, an internal tube placed inside the external tube such that a fluid passage is formed between the internal tube and the external tube, the internal tube having open portions on both ends, and an expandable member surrounding one end of the internal tube and being in fluid communication with the fluid passage, wherein one end of the expandable member is connected to the external tube and the other end of the expandable member is connected to the internal tube.

The internal tube may be extended to a terminal end of the expandable member.

A fitting portion may be connected to the external tube, and the internal tube may be pierced through the fitting portion.

The expandable member may be reduced in a spiral form before being expanded, and returned to the spiral form from an expanded form as fluid is released out.

A marking portion may be formed on one end of the internal tube at a predetermined distance that corresponds to an expandable length of the expandable member.

The bone may be a vertebra.

According to one embodiment, a device set comprising a device for fixation of bone, may include a guide wire, a dilator having a first piercing opening through which the guide wire is inserted, a cannula having a second piercing opening through which the dilator is inserted, and the device for fixation of bone according to one of claims 1 to 7, wherein the device for fixation of bone may be inserted through the second piercing opening of the cannula, and the guide wire may be inserted through an internal tube of the device for fixation of bone.

The device set may additionally include a needle assembly comprising a needle having a third piercing opening, and a needle pin inserted through the third piercing opening, and a drill comprising a fourth piercing opening through which the guide wire is inserted, and insertable through the second piercing opening of the cannula, wherein the guide wire may be inserted through the third piercing opening of the needle.

The dilator may be inclined so that a terminal end thereof has a gradually decreasing outer diameter, whereby the dilator reduces resistance of a human body when inserted.

The bone may be a vertebra.

According to the embodiments, unlike the conventional kyphoplasty which requires additional tools like stylet, since only the required components are included, the structure can be simple and manufacture cost can be reduced. Furthermore, since it is not necessary to additionally carry out a procedure of removing stylet in the procedure of forming a cavity in bone, the procedure can be simpler.

Furthermore, as the device for fixation of bone is inserted through the cannula to form a cavity in the vertebra, the presence of a guide wire inserted in the vertebra supports the expandable member so that the expandable member is not easily bent and inserted efficiently into the vertebra.

Furthermore, the simpler procedure reduces time for procedure and helps the patient to recover faster.

### BRIEF DESCRIPTION OF THE DRAWINGS

The above and/or other aspects of the present inventive concept will be more apparent by describing certain exemplary embodiments of the present inventive concept with reference to the accompanying drawings, in which:

FIG. 1 illustrates a device for fixation of bone according to an embodiment;

FIG. 2 illustrates an expansion member of a device for fixation of bone according to one example;

FIG. 3 illustrates a stage in which a needle assembly is inserted into a vertebra;

FIG. 4 illustrates a stage in which a needle pin is removed, while keeping the needle in a fixed state;

FIG. 5 illustrates a stage in which a guide wires is passed through a piercing opening of the needle and inserted into the vertebra;

FIG. 6 illustrates a stage in which the needle is removed along the guide wire;

FIG. 7 illustrates a stage in which a dilator and a cannula are inserted along the guide wire;

FIG. 8 illustrates a stage in which the dilator is removed from the interior of the cannula through the piercing opening of the cannula, to apply the device for fixation of bone according to one embodiment;

FIG. 9 illustrates a stage in which a drill is inserted into the cannula along the guide wire; and

FIG. 10A illustrates a stage in which the device for fixation of bone according to one embodiment is inserted through the piercing opening of the cannula, and FIG. 10B is a view of the expandable member of FIG. 10A in enlargement.

### DETAILED DESCRIPTION OF EXEMPLARY EMBODIMENTS

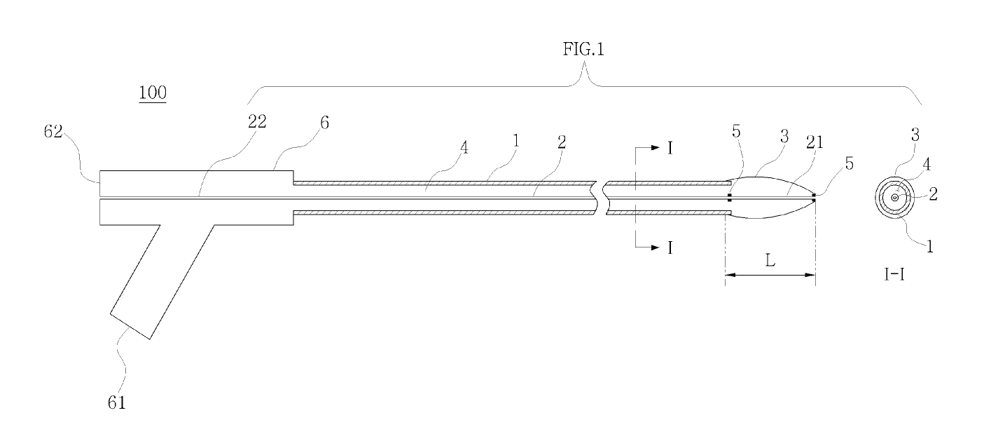

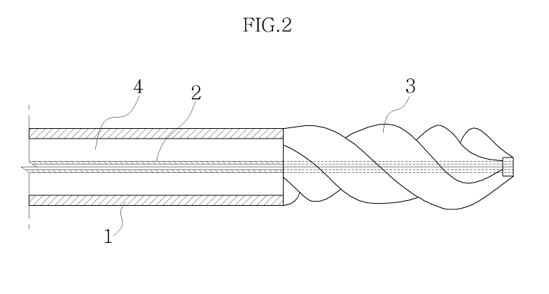

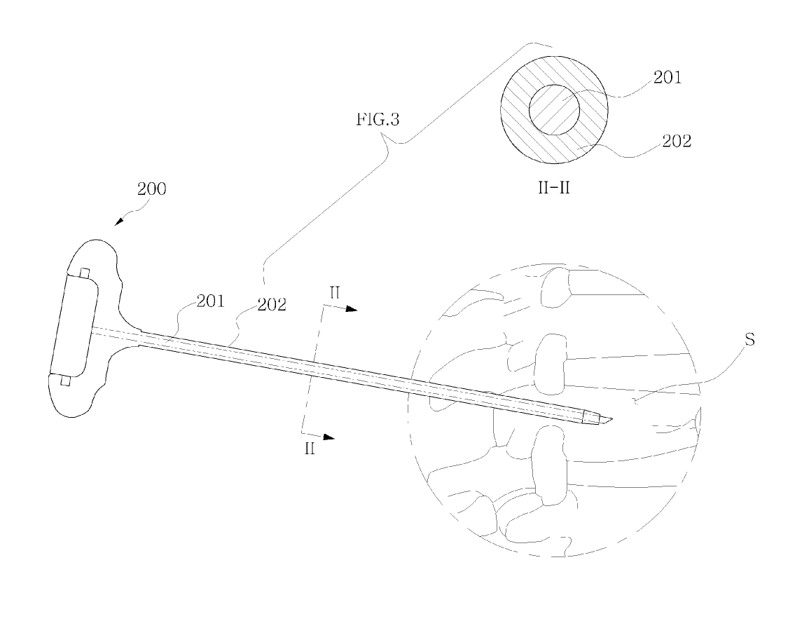

A device for fixation of bone according to certain exemplary embodiments of the present inventive concept will now be described in greater detail with reference to FIGS. 1 and 2.

In the following description, an example where a cavity is formed within vertebra is explained for illustrative purpose, but the embodiments of the present inventive concept are not limited to the specific example. Accordingly, the embodiments are applicable to procedures to form a cavity within various types of bones.

Also in the following description, same drawing reference numerals are used for the same elements even in different drawings. The matters defined in the description, such as detailed construction and elements, are provided to assist in a comprehensive understanding of the present inventive concept. Accordingly, it is apparent that the exemplary embodiments of the present inventive concept can be carried out without those specifically defined matters. Also, well-known functions or constructions are not described in detail since they would obscure the invention with unnecessary detail.

Referring to FIG. 1, a device 100 for fixation of bone according to one embodiment may include an external tube 1, an internal tube 2, and an expandable member 3.

The external tube 1 and the internal tube 2 may be in a tubular form, and made of high polymer material including, but not limited to, vinyl, polyethylene, polyamide, or polyurethane. The term ‘tubular form’ herein may refer to a cylindrical form with a predetermined wall thickness, which has a common hollow hole formed therein, and open portions formed on both ends.

The internal tube 2 may be placed within the external tube 1 such that a fluid passage 4 is formed between the internal tube 2 and the external tube 1. The internal tube 2 may have open portions on both ends. The material for the internal tube 2 may additionally include polymer or stainless steel to increase the stiffness and thus facilitate the insertion of the internal tube 2 into the vertebra through a cannula in which the expandable member 3 is inserted in the vertebra, during the balloon kyphoplasty for example.

A first end 21 of the internal tube 2 may be extended to the terminal end of the expandable member 3 to support the expandable member 3, and a second end 22 may be extended to one end 62 of a fitting portion 6 which will be explained in detail below. With respect to the first end 21 of the internal tube 2 in particular, it is possible to prepare the first end 21 by adding a stiffness improving substance such as polymer, nickel, titanium-based alloy, or stainless steel, so that the expandable member 3 is easily inserted into the vertebra through the cannula in the vertebra without having a bend during the procedure.

A marking portion 5 may be mounted on the first end of the internal tube 2 at a similar distance as the expandable length L of the expandable member 3. A doctor may use the marking portion 5 to determine the location of the expandable member 3 using radioactive equipment, while the device 100 according to the embodiment is inserted into the vertebra of the patient. Of course, the marking portion 5 may be formed on other proper places if occasion demands.

The expandable member 3 may surround the first end 21 of the internal tube 2. One and the other ends of the expandable member 3 may be connected to the external tube 1 and the internal tube 2, and the expandable member 3 may be in fluid communication with the fluid passage 4. The expandable member 3 may be connected to the internal and external tubes 2, 1 through processing such as thermal bonding.

Referring to FIG. 1, the expandable member 3 may be prepared in the oval, circular, sandglass, peanut, or asymmetric form. Referring to FIG. 2, the expandable member 3, when deflated, may be in spiral form. Accordingly, after expansion, as the fluid of the expandable member 3 is released, the expandable member 3 may be returned to the original spiral form. Due to such reduced form, the expandable member 3 takes up a little space and thus is able to be inserted efficiently into the expandable member 3 through the cannula in place within bone, or removed from the cannula after the formation of a cavity. The expandable length L of the expandable member 3 may be adjusted appropriately, depending on the length L between the terminal end of the external tube 1 and the terminal end of the internal tube 2.

The expandable member 3 may be inflated by the fluid introduced into the expandable member 3 through a fluid inlet 61 and a fluid passage 4. The expandable member 3 may be a balloon made of polymer including, but not limited to, synthetic resin, elastomer, or polyurethane. The fluid introduced into the expandable member 3 may include, but not limited to, air, contrast medium, or sterilized water.

The fitting portion 6 may be connected to the external tube 1 and the internal tube 2 may be pierced through the fitting portion 6 and fixed to the one end 62 of the fitting portion 6. The doctor grabs the fitting portion 6 as a handle during the procedure.

The fluid inlet 61 may be formed on a side of the fitting portion 6. The fluid inlet 61 may be connected to a supply device (not illustrated) which supplies the fluid such as air, contrast medium, or the like. Referring to FIG. 1, the fluid inlet 61 is separately formed on a side of the fitting portion 6, forming Y shape. However, depending on occasions, the fluid inlet 61 may be formed on the one end 62 of the fitting portion 5, instead of being formed on the side of the fitting portion 5, to thus increase simplicity of the shape of the fitting portion 5.

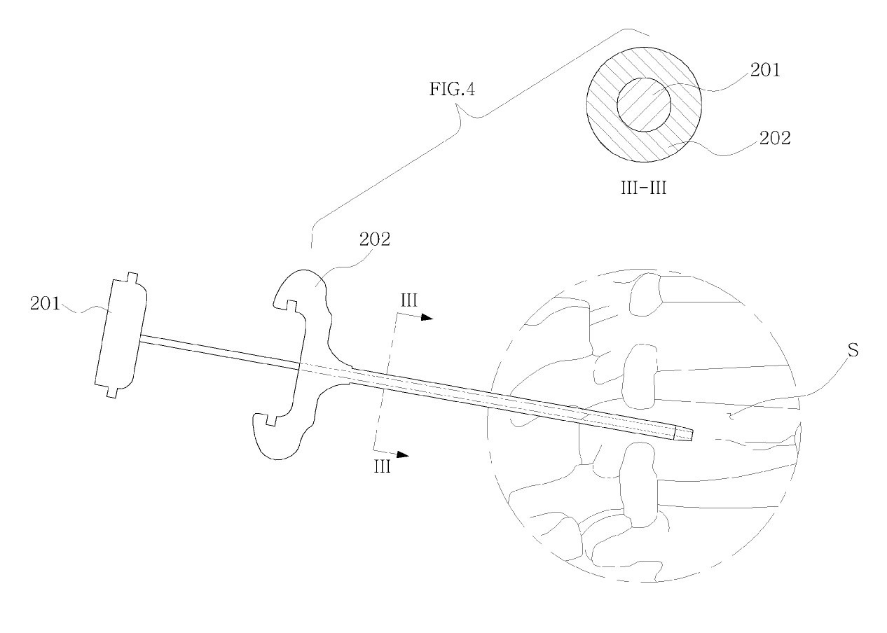

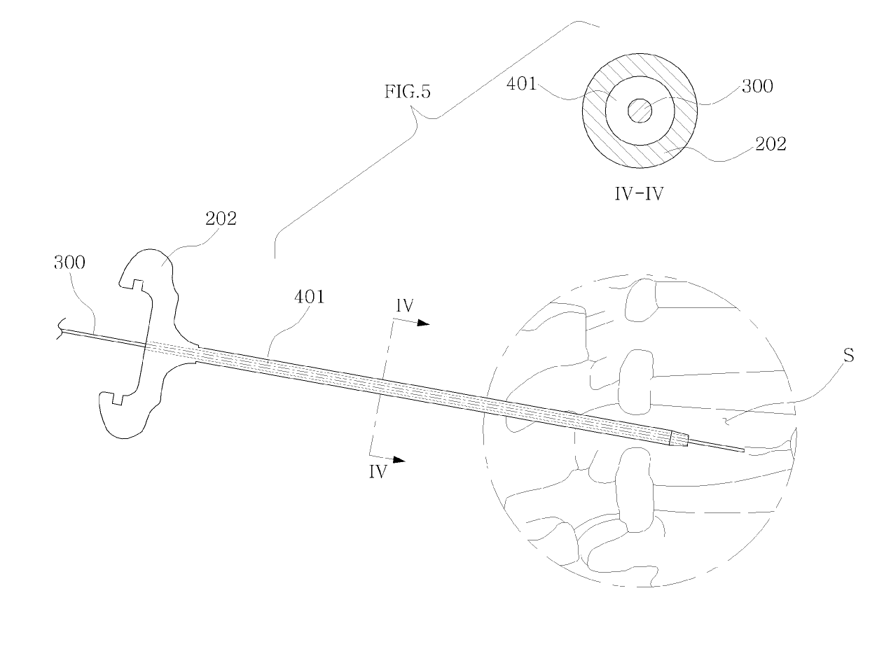

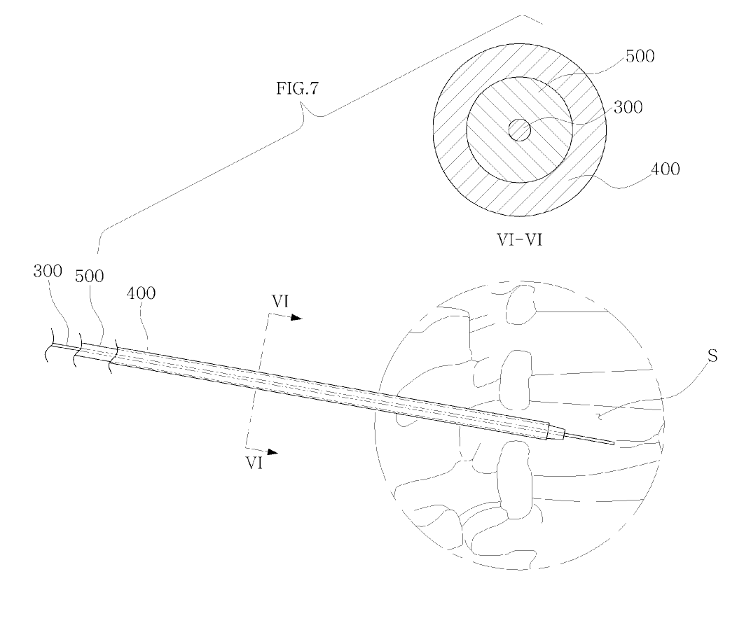

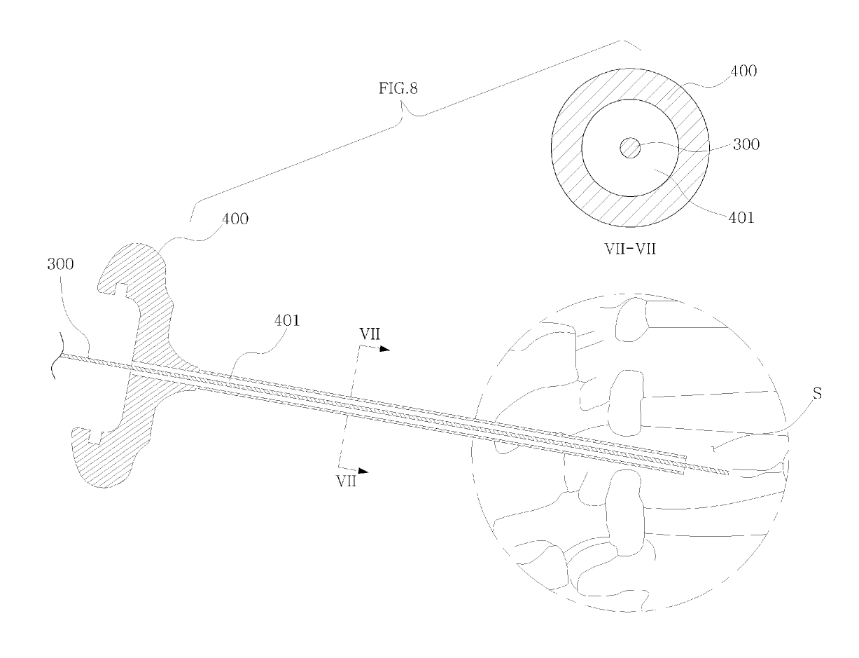

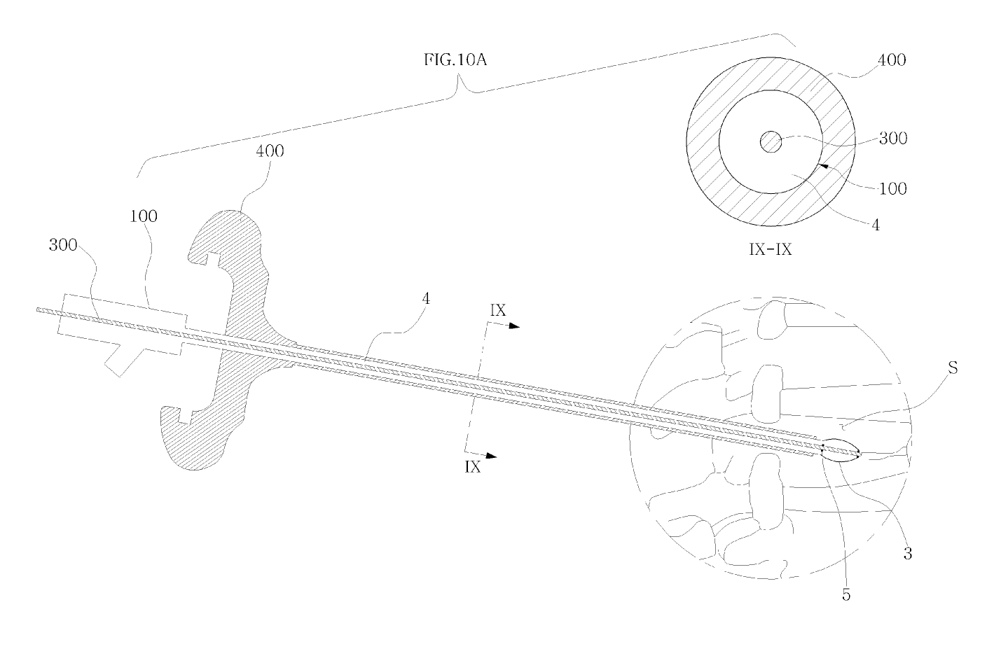

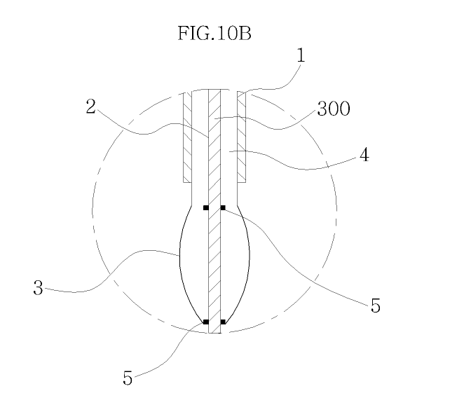

Hereinbelow, a device set including the device for fixation of bone according to an embodiment, and an exemplary method for conducting balloon kyphoplasty using the device set will be explained. In the following description, the exemplary method will be explained only in relation to the main idea of the present inventive concept, and accordingly, others that are considered irrelevant to the present inventive concept will not be explained for the sake of brevity. Also in the following description, while the cross-section views taken along line II-II in FIG. 3, line III-III in FIG. 4, line VI-VI in FIG. 7, line VIII-VIII in FIG. 9, and line IX-IX in FIG. 10A illustrate that the members are inserted within the neighboring members without having a gap therebetween, one will understand that a slight space may be formed between the neighboring members.

First, referring to FIG. 3, at S1, the doctor inserts a needle assembly 200 into the patient's vertebra S (marrow, for example) of a patient with CT or radiological observation. The needle assembly 200 may include a needle 202 which has a piercing opening (third opening) formed therein, and a needle pin 201 to be inserted through the piercing opening of the needle 202. The terminal ends of the needle 202 and the needle pin 201 are pointed sharply to be able to pierce into the tissues of the patient.

Next, referring to FIG. 4, at S2, the doctor removes the needle pin 201 from the piercing opening of the needle 202, while holding the needle 202 in a fixed state.

Referring to FIG. 5, at S3, the doctor prepares a guide wire 300, and slides the guide wire 300 through the piercing opening of the needle 202 so that the guide wire 300 is inserted into the vertebra (S). The guide wire 300 may be prepared in a slender bar shape, and made of a metal material such as stainless steel and a polymer material with stiffness. The guide wire 300 is used to set the path and direction to the subject vertebra (S) of treatment.

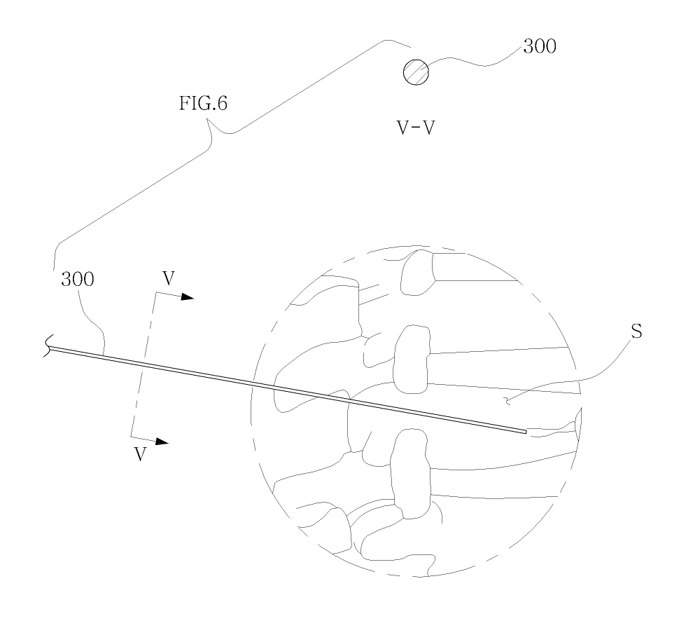

Referring to FIG. 6, at S4, the doctor removes the needle 202 along the guide wire 300, and leaves the guide wire 300 in the inserted state, i.e., leaves the guide wire 300 in the vertebra S.

Referring to FIG. 7, at S5, the doctor inserts the cannula 400 with a dilator 500 formed therein, along the guide wire 300. The cannula 400 includes a piercing opening (second opening) through which the dilator 500 is inserted. The dilator 500 includes a piercing opening (first opening) through which the guide wire 300 is inserted. Meanwhile, the outer diameter of the terminal end of the cannula 400 is so set that it is difficult to insert the cannula 400 against the resistance of the patient's body. However, since the terminal end of the dilator 500 is inclined to have gradually decreasing outer diameter, this helps to decrease the resistance by the patient's body and thus facilitate the insertion of the cannula 400 into the patient's body.

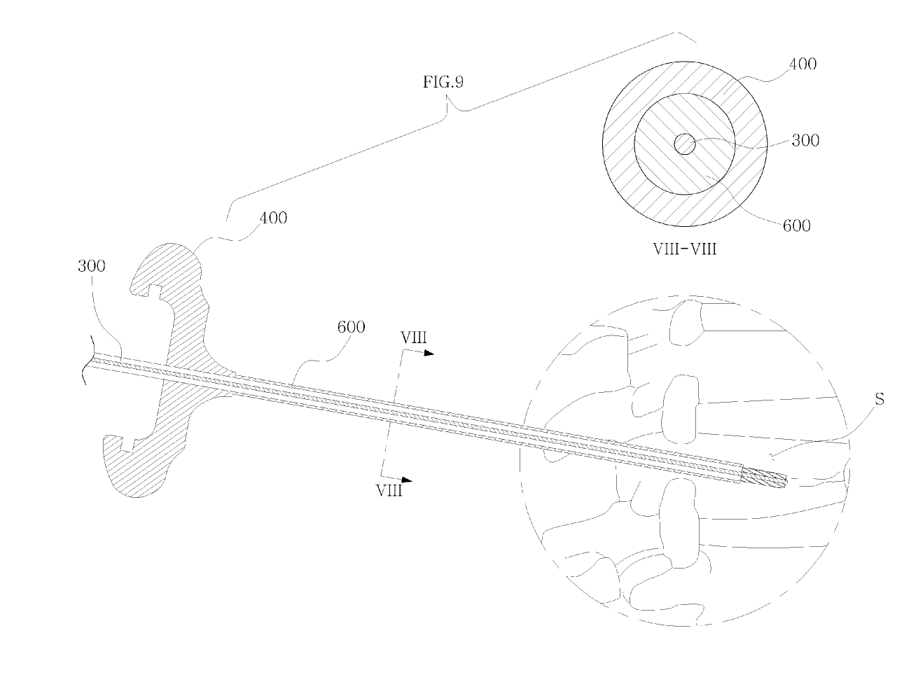

Referring to FIG. 8, at S6, the doctor removes the dilator 500 from the interior of the cannula 400 along the piercing opening 401 of the cannula 400. In this situation, the guide wire 300 remains in the cannula 400.

Referring to FIG. 9, at S7, the doctor inserts a drill 600 having therein a piercing opening (fourth opening) through which the guide wire 300 is inserted, through the piercing opening 401 of the cannula 400, and drills the vertebra S to a predetermined depth using the inserted drill 600. The space formed by the drilling is used later as a passage through which the expandable member 3 is entered.

Referring to FIGS. 10A and 10B, at S8, the doctor removes the drill 600 from the piercing opening 401 of the cannular 400, while maintaining the guide wire 300 in the current state, and inserts the device 100 for fixation of bone according to an embodiment through the piercing opening 401 of the cannula 400, in which the device 100 is slid along the guide wire 300 as the guide wire 300 is inserted into the tube 2 formed within the device 100, and the expandable member 3 of the device 100 is placed on the subject vertebra S of the treatment.

At S9, as the fluid supply device (not illustrated) is connected to the fluid inlet 61 and the fluid is supplied, the expandable member 3 is inflated, thereby compressing the vertebra S and forming a cavity within the vertebra S into which a filler material such as orthopaedic cement is to be injected.

Next, the doctor injects orthopaedic cement into the cavity of the vertebra S using a filler material injecting device (not illustrated). Depending on occasions, additional procedure(s) may be added between, before or after the procedures explained above. These additional procedures, however, are considered irrelevant to the main idea of the present inventive concept, and therefore will not be explained in detail.

Meanwhile, the guide wire 300, which is inserted into the vertebra S at S3 and remains in the inserted state from then on, provides the internal and external tubes 2, 1 and the expandable members 3 with necessary stiffness when the device 100 for fixation of bone is inserted into the vertebra S through the cannula 400. That is, the guide wire 300 keeps the internal and external tubes 2, 1 and the expandable member 3 from bending so as to support the expandable member 3 and facilitate the insertion of the expandable member 3 into the vertebra S through the cannula 400.

Accordingly, compared to the conventional treatment which requires the use of a separate stylet to maintain the expandable member (i.e., balloon) in a correct state during insertion of a catheter tube with the expandable member through the cannular inserted in the vertebra, and the removal of the stylet after use, the embodiments of the present inventive concept resolves the shortcomings of the conventional treatment. Accordingly, using the device for fixation of bone and the device set comprising the device for fixation of bone according to the embodiments of the present inventive concept, the balloon kyphoplasty with increased ease and simplicity can be provided.

The foregoing exemplary embodiments and advantages are merely exemplary and are not to be construed as limiting the present invention. The present teaching can be readily applied to other types of apparatuses. Also, the description of the exemplary embodiments of the present inventive concept is intended to be illustrative, and not to limit the scope of the claims, and many alternatives, modifications, and variations will be apparent to those skilled in the art.

What is claimed is:

1. A device for fixation of bone, which forms a cavity in bone, the device comprising:
an external tube;
an internal tube placed inside the external tube such that a fluid passage is formed between the internal tube and the external tube, the internal tube having open portions on both ends; and
an expandable member surrounding one end of the internal tube and being in fluid communication with the fluid passage, wherein one end of the expandable member is connected to the external tube and the other end of the expandable member is connected to the internal tube.

2. The device of claim 1, wherein the internal tube is extended to a terminal end of the expandable member.

3. The device of claim 1, wherein a fitting portion is connected to the external tube, and the internal tube is pierced through the fitting portion.

4. The device of claim 3, wherein a fluid inlet is formed in the fitting portion, and the fluid inlet is in fluid communication with the fluid passage.

5. The device of claim 1, wherein the entirety or one end of the internal tube comprises a stiffness improving material.

6. The device of claim 1, wherein the expandable member is reduced in a spiral form before being expanded, and returned to the spiral form from an expanded form as fluid is released out.

7. The device of claim 1, wherein a marking portion is formed on one end of the internal tube at a predetermined distance that corresponds to an expandable length of the expandable member.

8. The device of claim 1, wherein the bone is a vertebra.

9. A device set comprising a device for fixation of bone, the device set comprising:
a guide wire;
a dilator having a first piercing opening through which the guide wire is inserted;
a cannula having a second piercing opening through which the dilator is inserted, and
the device for fixation of bone according to claim 1,
wherein the device for fixation of bone may be inserted through the second piercing opening of the cannula, and the guide wire may be inserted through an internal tube of the device for fixation of bone.

10. The device set of claim 9, further comprising:
a needle assembly comprising a needle having a third piercing opening, and a needle pin inserted through the third piercing opening; and
a drill comprising a fourth piercing opening through which the guide wire is inserted, and insertable through the second piercing opening of the cannula,
wherein the guide wire may be inserted through the third piercing opening of the needle.

11. The device set of claim 9, wherein the dilator may be inclined so that a terminal end thereof has a gradually decreasing outer diameter, whereby the dilator reduces resistance of a human body when inserted.

12. The device set of claim 9, wherein the bone is a vertebra.
